(19) United States
(12) Patent Application Publication
(10) Pub. No.: US 2007/0231882 A1
(43) Pub. Date: Oct. 4, 2007

(54) MEASUREMENT APPARATUS AND ELEMENT FOR ANALYSIS

(76) Inventors: Masao Kamahori, Kokubunji (JP); Yu Ishige, Tokyo (JP)

Correspondence Address: MATTINGLY, STANGER, MALUR & BRUNDIDGE, P.C., 1800 DIAGONAL ROAD, SUITE 370, ALEXANDRIA, VA, 22314, US

(21) Appl. No.: 11/617,230

(22) Filed: Dec. 28, 2006

(30) Foreign Application Priority Data
Mar. 30, 2006 (JP) 2006-092950

(51) Int. Cl. C12M 3/00; H01L 51/00

(52) U.S. Cl. 435/287.2; 257/40

(57) ABSTRACT

The production amount of thiol compounds which is the product of the cyclic reaction of enzyme-labeled antibody, or the production rate thereof is measured as an adsorption rate on a gold electrode formed on an insulated gate field-effect transistor. The adsorption rate is measured by monitoring in real time the change in a potential on the gold electrode associated with the formation of a self-assembled monolayer on the gold electrode, that is, the current between a source and a drain in the insulated gate field-effect transistor. The measured adsorption rate is recorded by using a signal processing circuit and a data processing unit. Then, the amount of antigen is found from the adsorption rate. During this measuring, a high frequency voltage is applied to a reference electrode from a power supply to reduce the effect of external variations of the measurement.

### CLAIM OF PRIORITY

The present application claims priority from Japanese application JP 2006-092950 filed on Mar. 30, 2006, the content of which is hereby incorporated by reference into this application.

### BACKGROUND OF THE INVENTION

1. Field of the Invention

The present invention relates to a measurement apparatus which allows highly sensitive measurement of a biomaterial such as protein, and to an element for analysis thereof.

2. Description of the Prior Art

An enzyme immunoassay method, which is a typical method of measuring protein, utilizes a reaction in which antibody selectively binds to specific protein, that is, an antigen-antibody reaction. As a measuring method, there are a sandwich method and a competitive reaction method. In the sandwich method, the amount of antigen is indirectly measured by using enzyme with which antibody is labeled. After primary antibody previously immobilized to a solid phase reacts with antigen in a sample, enzyme-labeled antibody is added to the reaction product to form primary antibody-antigen-enzyme-labeled antibody complex. Then, the bound enzyme-labeled antibody (B) and free enzyme-labeled antibody (F) are separated from each other (BF separation). Subsequently, by measuring the luminescence or the absorbance of the product of a cyclic reaction between the enzyme of the bound enzyme-labeled antibody (B) and the substrate, the amount of the antigen is obtained. The competitive reaction method utilizes binding properties of unlabeled antigen to be measured and the enzyme-labeled antigen having a known concentration, which competitively bind to the immobilizing antibody. The amount of the enzyme-labeled antigen bound to the antibody depends on the concentration ratio of the antigen to be measured to the enzyme-labeled antigen. Accordingly, the amount of the antigen to be measured can be determined by measuring the amount of the bound enzyme-labeled antigen. In particular, the sandwich method has such a high sensitivity as to be currently widely used. The sandwich method is also used for measuring peptide and protein which are a marker of disease and present in a body in extremely minute amounts.

With increasing health consciousness in recent years, the countermeasure and prophylaxis against life-style related diseases have become a matter of concern. Specifically, a heart disease and a cerebrovascular disease are a major cause of death second only to cancer. Hence, the prophylaxis against them becomes more important. For example, brain natriuretic peptide (BNP) has a vasodilating effect and a diuretic effect, and plays an important role in the adjustment of the amount of a body fluid and a blood pressure. The BNP concentration in the blood plasma of a healthy person is extremely low. However, the value of a patient who has heart failure is increased according to the degree of severity. Accordingly, the measurement of the BNP concentration is of great significance to understand a clinical state of the heart failure. The BNP concentration of a healthy person is 18 ppt (pg/mL). Thus, a measurement sensitivity of 10 ppt or less is required of a measurement apparatus. It is said that TNF-α (Tumor Necrosis Factor), which is one of inflammatory cytokines, also plays an extremely significant role in damaging the heart muscle, and particularly causing heart failure. The level of TNF-α is increased in the blood or the heart tissue of a patient who has heart failure. This increase in level is highly responsible for the development of the pathological condition of the heart failure. The concentration is at an extremely low level of 1 ppt or less. The cytokine is multifunctional protein which takes an important role in the development and adjustment of a biological reaction against a disease and infection, and particularly is an important factor for controlling immunity and inflammation. For example, alcoholic hepatitis, hepatitis, rheumatoid arthritis, spinal cord disease, and cephalopathy are known as a disease in which the concentration of interleukin-1β is increased. The concentration of interleukin-1β of a healthy person is 10 ppt or less.

In the above background, as a simple and easy method of measuring substances, which exist in a body in extremely minute amounts, with high sensitivity, an electrochemical enzyme immunoassay method has been proposed which is the combination of enzyme immunoassay method and electrochemical detection method. In the conventional enzyme immunoassay method using the sandwich method, after the reaction of primary antibody previously immobilized to a solid phase to the antigen in a sample, enzyme-labeled antibody is added to the reaction product to form primary antibody-antigen-enzyme-labeled antibody complex. Then, the bound enzyme-labeled antibody (B) and the free enzyme-labeled antibody (F) are separated from each other (BF separation). Subsequently, by measuring the luminescence or the absorbance of the product of a cyclic reaction between the enzyme of the bound enzyme-labeled antibody (B) and the substrate, the amount of the antigen is obtained. In the electrochemical enzyme immunoassay method, the same processes as those of the conventional method are performed until the BF separation, but the amount of the antigen is electrochemically measured by adsorbing and concentrating, to on a silver electrode, the product of the cyclic reaction of the enzyme-labeled antibody (B). At the time of this measurement, cholinesterase is used as enzyme which is used to label the antibody. Thiocholin, which is a substance decomposed by cholinesterase, is adsorbed on the silver electrode and concentrated thereon. Subsequently, the amount of the antigen is detected by measuring a current signal generated due to reduction desorption, in strong alkaline solution, of the thiocholin adsorbed on the silver electrode (Japanese Patent Application Publication No. 2004-257996). In this way, a minute amount of an enzyme reaction product can be concentrated on the silver electrode, so that highly sensitive measurement can be performed. The present method is an application of the reduction desorption of thiol compounds bound to the surface of gold (Langmuir 7, (1991) 2687-2693). Another example of the application of this reduction desorption is described in a report on the measurement of the activity of acetylcholinesterase (Sensors and Actuators B 91, (2003) 148-151).

### SUMMARY OF THE INVENTION

In the electrochemical enzyme immunoassay method using an electrochemical method, it is necessary to perform the reduction desorption reaction of the thiocholin adsorbed on a silver electrode in a strong alkaline solution (for example, 0.5 M KOH solution). Thus, in an electrochemical measurement method, it is necessary to replace the solution used for performing the processes until the BF separation with the strong alkaline solution. The use of the strong alkaline solution presents problems in safety, and therefore it should be handled with care. In addition, in the present measurement method, the thiocholin which is the enzyme reaction product of cholinesterase is measured after adsorbed on the silver electrode. Accordingly, the measurement is performed at the endpoint of the enzyme reaction. Thus, a direct measurement of the reaction rate of the enzyme is impossible. That is, there is a problem of the reduction in measurement accuracy because the intermediary state of the enzyme reaction of cholinesterase cannot be measured. Even in the method of reduction-desorbing the thiocholin adsorbed on the silver electrode, there is a problem that the desorption is so easily affected by foreign substances that the baseline is difficult to be stabilized, resulting in the reduction in measurement accuracy because the amount of the adsorbed thiocholin is estimated from the peak area of the reduction current.

It is an object of the present invention to provide a highly sensitive and high accuracy measurement apparatus and a measurement method which can be used without replacing the solution and in convenience, and which allows a direct measurement of enzyme reaction rate of the labeling enzyme.

In the present invention, the production amount or production rate of thiol compounds which is the reaction product of the cyclic reaction of enzyme-labeled antibody is measured by means of a field-effect transistor to achieve the above object. The field-effect transistor has, for example, a gold electrode in a sensing section, and is connected with a gate of an insulated gate field-effect transistor via an electroconductive wire. The change of potentials associated with the adsorption of the thiol compounds on the gold electrode in the sensing section is measured as the change of a drain current between a source and a drain of the field effect transistor. That is, an enzyme reaction in which the thiol compounds are produced is performed in the same container. The adsorption reaction of the produced thiol compounds on the gold electrode is measured as the change of the drain current. Even in a case where the enzyme reaction in which the thiol compounds are produced, and the adsorption reaction of the formed thiol on the gold electrode are performed in the different containers, after the solution in the container containing the produced thiol compounds is transferred to the container in which the adsorption reaction to the gold electrode is performed, the amount of the thiol compounds is measured as the change in the drain current in the same manner as above. At the time of the measurement, an alternating current will be applied between the gold electrode and a reference electrode. Furthermore, to reduce a drift during measurement, straight-chain polymer is preferably used as being physically adsorbed on the gold electrode.

According to the present invention, the change of the drain current associated with the adsorption of the thiol compounds on the gold electrode is measured by using the field-effect transistor having the gold electrode. The thiol compounds are the reaction product of the cyclic reaction of the enzyme which is used for labeling the antibody. Then, the production amount or production rate of the thiol compounds can be measured. At this time, if the enzyme reaction in which the thiol compounds are produced and the adsorption reaction of the produced thiol compounds on the gold electrode are performed in the same container, the enzyme reaction can be measured in real time, resulting in the achievement of high accuracy measurement. Even in a case where the enzyme reaction in which the thiol compounds are produced, and adsorption reaction of the produced thiol compounds on the gold electrode are performed in different containers, the amount of the produced thiol compounds can be measured by performing a simple operation only in which the solution in the container containing the produced thiol compounds is transferred to the container in which the adsorption reaction on the gold electrode is performed. Accordingly, the operations until the B/F separation and the enzyme reaction in a conventional method can be performed, and a new operation is not necessary for the measurement of the present invention to be performed. The adsorption of foreign substances on the gold electrode and the effect of the drift by, for example, ions in the solution which are troublesome in using the gold electrode in the solution can easily be removed by applying an alternating voltage between the gold electrode and the reference electrode. Alternatively, the drift can be reduced by physically adsorbing the straight-chain polymer on the gold electrode.

### BRIEF DESCRIPTION OF THE DRAWINGS

FIG. 1 is a block diagram showing an example of an immuno analyzer according to the present invention.

FIGS. 2A and 2B are drawings showing an example of the structure of an insulated gate field-effect transistor used in the immuno analyzer according to the present invention, FIG. 2A is a cross-sectional view, and FIG. 2B is a plan view.

FIG. 3 is a drawing showing an example of the immuno analyzer according to the present invention.

FIG. 4 is a block diagram showing an example of an immuno analysis system according to the present invention.

FIG. 5 is a drawing showing a reaction flow of the immuno analysis according to the present system.

FIGS. 6A to 6H are drawings showing an effect of the high frequency superposition method according to the present invention (frequencies of the applied voltage, FIG. 6A: DC, FIG. 6B: 10 HZ, FIG. 6C: 100 Hz, FIG. 6D: 1 KHz, FIG. 6E: 10 KHz, FIG. 6F: 100 KHz, FIG. 6G: 1 MHz, and FIG. 6H: 10 MHz).

FIGS. 7A to 7H are drawings showing measurement results of thiol compound solutions having different concentrations (concentrations of the thiol compounds, FIG. 7A: 50 μM, FIG. 7B: 25 μM, FIG. 7C: 10 μM, FIG. 7D: 5 μM, FIG. 7E: 2.5 μM, FIG. 7F: 0.5 μM, and FIG. 7G: 0.1 μM).

FIG. 8 is a drawing showing the relationship between the concentration of the thiol compounds and the adsorption rate on a gold electrode according to the present invention.

FIGS. 9A to 9E are drawings showing measurement results by means of the immuno analyzer according to the present invention.

FIG. 10 is a drawing showing the relationship between the concentration in the sample solution and the adsorption rate which are measured by an apparatus according to the present invention.

FIG. 11 is a drawing showing the relationship between the concentration in the sample solution and the adsorption rate which are measured by using the apparatus according to the present invention.

FIG. 12 is a drawing showing an effect of the straight-chain polymer which is physically adsorbed on the gold electrode.

FIG. 13 is a drawing showing an effect of the straight-chain polymer which is physically adsorbed on the gold electrode.

FIGS. 14A to 14C are drawings showing an effect in a case of the combination of the gold electrode on which the straight-chain polymer is physically adsorbed and the high frequency superposition method.

FIG. 15 is a drawing showing the relationship between the frequency of the applied alternating voltage and the change (ΔV) in an interface potential.

### DESCRIPTION OF THE PREFERRED EMBODIMENTS

With reference to the drawings, the embodiments of the present invention will hereinafter be described.

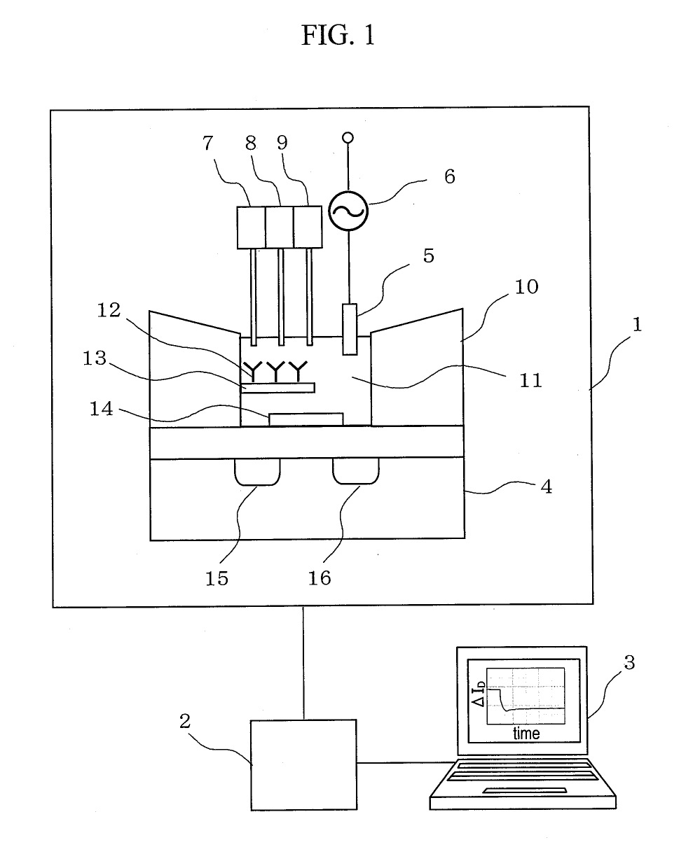

FIG. 1 is a block diagram showing an example of an immuno analyzer using an FET sensor according to the present invention. A measurement system of the present invention is configured of a measurement section 1, a signal processing circuit 2, and a data processing unit 3. The measurement section 1 is provided with an insulated gate field-effect transistor 4, a reference electrode 5, a power supply 6 for applying a high frequency voltage to the reference electrode 5, a sample solution injector 7 for supplying the sample solution containing a substance to be measured, an enzyme-labeled antibody solution injector 8 for supplying the enzyme-labeled antibody which is formed by combining enzyme which produces thiol compounds with antibody to the substance to be measured, a substrate solution injector 9 for supplying the substrate of the enzyme which produces the thiol compounds, and a measurement cell 10. In a reaction solution 11 in the measurement cell 10, an antibody immobilizing plate 13 on which antibody 12 is immobilized, a gold electrode 14 formed on the insulated gate field-effect transistor 4, and the reference electrode 5 are disposed.

A measurement procedure is described below. First, the sample solution is injected in the reaction solution 11 in the measurement cell 10 using the sample solution injector 7 to bind antigen in the sample solution to the antibody 12. After a certain time, the enzyme-labeled antibody solution is injected in the reaction solution 11 using the enzyme-labeled antibody solution injector 8 to cause an antigen-antibody reaction for formation of antibody-antigen-enzyme-labeled antibody complex. Then, the bound enzyme-labeled antibody and free enzyme-labeled antibody are separated from each other by cleaning the measurement cell 10 and by the replacing the reaction solution 11 in the measurement cell 10. After cleaning the measurement cell 10 and replacing the reaction solution, when the substrate of the labeling enzyme is injected using the substrate solution injector 9, the substrate is decomposed by the enzyme to form the thiol compounds. The formed thiol compounds are adsorbed on the gold electrode 14 provided on the insulated gate field-effect transistor 4 to form a self-assembled monolayer. As a result, the potential on the gold electrode 14 is changed. The measurement is performed by monitoring in real time the current between a source 15 and a drain 16 in the insulated gate field-effect transistor 4 which are changed before and after the injection of the substrate by means of the substrate solution injector 9, and by recording the monitored value using the signal processing circuit 2 and the data processing unit 3. The rate of the adsorption of the thiol compounds on the gold electrode 14 is proportional to the production rate of the thiol compounds, that is, to the amount of antibody-antigen-enzyme-labeled antibody complex. Accordingly, the amount of the bound labeling enzyme, that is, the amount of the antigen in the sample solution can be obtained by measuring the adsorption rate of the thiol compounds on the gold electrode 14. During this measuring, a high frequency voltage is applied to the reference electrode 5 from the power supply 6 to reduce the effect of external variations of measurement.

In the sample solution injector 7, the enzyme-labeled antibody solution injector 8, and the substrate solution injector 9, a syringe pump or a pressure type liquid transfer apparatus can be used. When the volume to be injected is 1 μL or more, both of the syringe pump or the pressure type liquid transfer apparatus can be used. When the volume to be injected is 1 μL or less, the pressure type liquid transfer apparatus using a capillary in a resistance tube is desirable. For example, in a case where the volume to be injected is 0.2 μL, injection can be performed with accuracy under conditions of pressure of 2 atmosphere and a pressing time of 2 seconds by using a flow control capillary having an inner diameter of 25 μm and a length of 20 mm.

The reference electrode 5 supplies a reference potential to constantly measure a potential variation based on equilibrium reaction or chemical reaction which occurs on the surface of the gold electrode 14 in the reaction solution 11. As a reference electrode, a silver-silver chloride electrode using a saturated potassium chloride solution as an inner solution, or calomel electrode is usually used. In a case where the composition of the sample solution to be measured is constant, the silver-silver chloride electrode only can be used as a quasi-electrode without a problem.

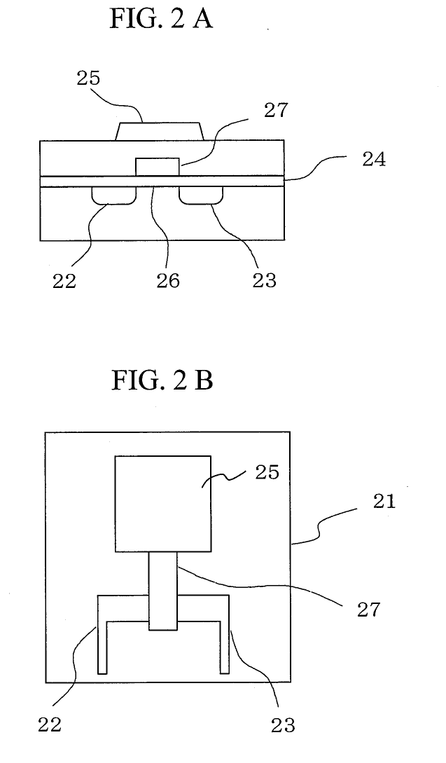

FIGS. 2A and 2B are drawings showing the structure of the insulated gate field-effect transistor used in the immuno analyzer according to the present invention. FIG. 2A and FIG. 2B show a cross sectional structure and a planar structure, respectively. An insulated gate field-effect transistor 21 is provided with a source 22, a drain 23, and a gate insulator 24 which are formed on the surface of a silicon substrate, and a gold electrode 25. The gold electrode 25 and the gate 26 of the insulated gate field-effect transistor are connected with each other via an electroconductive wire 27. The insulated gate field-effect transistor is preferably a metal-oxide semiconductor insulated field-effect transistor (FET) using a silicon oxide as an insulation film. A thin film transistor (TFT) can be used without a problem. Using the present structure, the gold electrode 25 can be formed in any place and in any size, and the volume of the measurement cell can be changed depending on the amount of the sample solution to be measured. The insulated gate field-effect transistor used in the present invention is a depletion type FET having an insulation layer using SiO₂ (thickness: 17.5 nm), and is provided with the gold electrode formed in a size of 400 μm×400 μm. In usual measurement, an aqueous solution is used. Accordingly, the present element is required to operate in a solution. In a case where the measurement is performed in a solution, the element is also required to operate within an electrode potential range of −0.5 V to 5 V that hardly causes an electrochemical reaction. Thus, in the present example, the manufacturing condition of the depletion type n-channel FET, that is, a threshold voltage (Vt) adjustment ion implantation condition is so adjusted that the threshold voltage of the FET is set around −0.5 V. Note that, an electrode made of other noble metal such as silver may be used instead of the gold electrode.

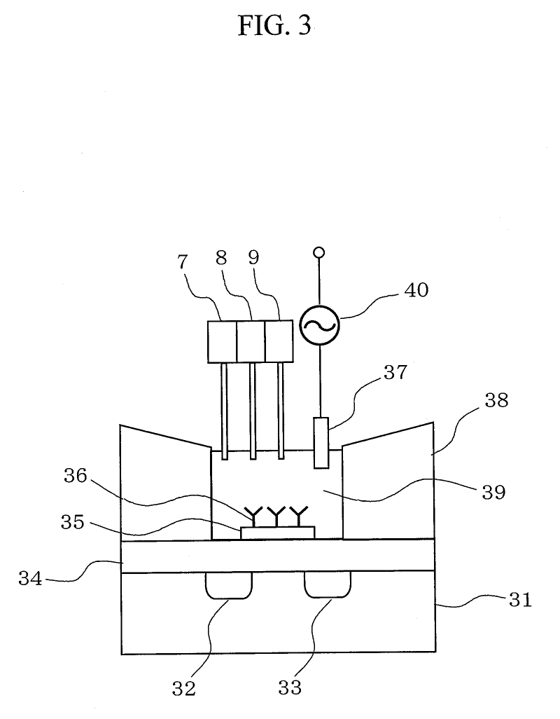

FIG. 3 is a drawing showing an example of another structure of an immuno analyzer using the FET sensor according to the present invention. An insulated gate field-effect transistor 31 used in the present example is provided with a source 32, a drain 33, and a gate insulator 34 which are formed on the surface of a silicon substrate, and a gold electrode 35 mounted on the surface of a gate insulator between the source 32 and the drain 33. Antibody 36 is immobilized on the surface of the gold electrode 35.

In actual measurement, the gold electrode 35, the antibody 36 immobilized on the surface of the gold electrode 35 and a reference electrode 37 are disposed in a reaction solution 39 in a measurement cell 38. Then, a high frequency voltage is applied to the reference electrode 37 from a power supply 40 to detect an enzyme reaction product formed in the reaction solution 39 as the change in the electric property of the insulated gate field-effect transistor 31 caused before and after the injection of the substrate, that is, as the change in current caused between the source 32 and the drain 33. Thus, the amount of the antigen bound to the antibody 36 in the sample solution can be measured.

A measurement procedure is described below. First, a sample solution is injected in the reaction solution 39 in the measurement cell 38 using the sample solution injector 7 to bind the antigen in the sample solution to the antibody 36. After a certain time, the enzyme-labeled antibody solution is injected in the reaction solution 39 using the enzyme-labeled antibody solution injector 8 to cause the antigen-antibody reaction for the formation of antibody-antigen-enzyme-labeled antibody complex. Then, the bound enzyme-labeled antibody and the free enzyme-labeled antibody are separated from each other by replacing the reaction solution 39 in the measurement cell 38 and by cleaning the measurement cell 38. After replacing the reaction solution 39 in the measurement cell 38 and cleaning the measurement cell 38, when the substrate of the labeling enzyme is injected using the substrate solution injector 9, the substrate is decomposed by the enzyme to form the thiol compounds. The formed thiol compounds are adsorbed on the gold electrode 35 provided on the insulated gate field-effect transistor 31 to form a self-assembled monolayer. As a result, the potential on the gold electrode 35 is changed. The rate of the adsorption of the thiol compounds on the gold electrode 35 is proportional to the production rate of the thiol compounds, that is, to the amount of the antibody-antigen-enzyme-labeled antibody complex. Accordingly, the amount of the bound labeling enzyme, that is, the amount of the antigen in the sample solution can be obtained by measuring the adsorption rate of the thiol compounds on the gold electrode 35. In this time, for the immobilization on the gold electrode 35, Fab′ fragment which is a part of antibody and an aptamer which is a single-chain DNA can be used in addition to antibody without a problem.

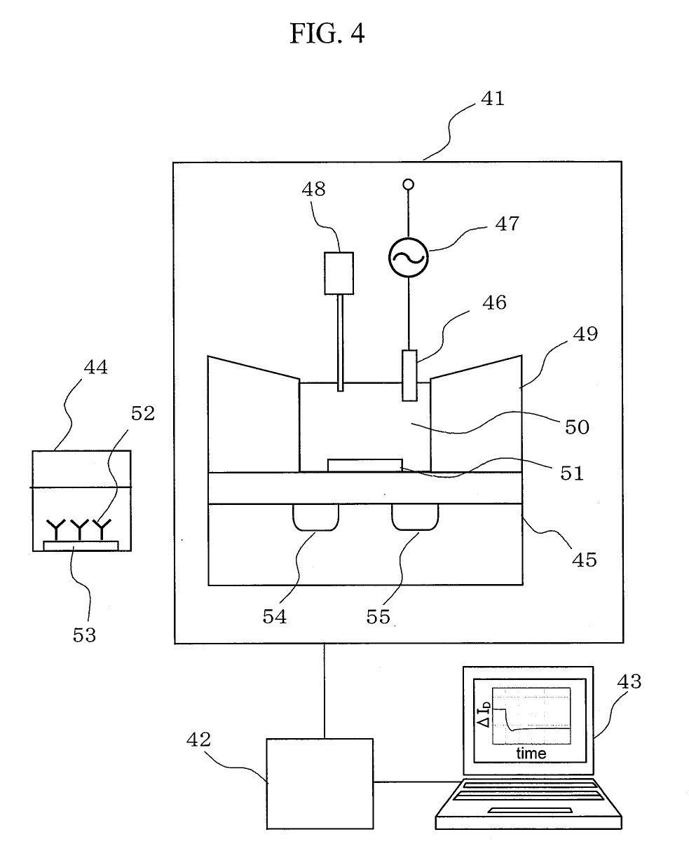

FIG. 4 is a block diagram showing an example of an immuno analysis system using the FET sensor according to the present invention. The analysis system is configured of a measurement section 41, a signal processing circuit 42, a data processing unit 43, and a reaction container 44 for the thiol compound production reaction. An insulated gate field-effect transistor 45, a reference electrode 46, a power supply 47 for applying high frequency voltage to the reference electrode 46, and a thiol compound solution injector 48 for supplying the solution in the reaction container 44 are provided in the measurement section 41. In a reaction solution 50 in a measurement cell 49, a gold electrode 51 formed on the insulated gate field-effect transistor 45, and the reference electrode 46 are disposed. Antibody 52 is immobilized on an antibody immobilizing plate 53 in the reaction container 44 for the thiol compound production reaction. Note that, the antibody 52 may be directly immobilized in the reaction container 44.

A measurement procedure is described below. A sample solution is injected in the reaction container 44 for the thiol compound production reaction to bind the antigen in the sample solution to the antibody 52. After a certain time, the enzyme labeled antibody solution is injected in the reaction container 44 to cause the antigen-antibody reaction for formation of the antibody-antigen-enzyme-labeled antibody complex. Then, the bound enzyme-labeled antibody and the free enzyme-labeled antibody are separated from each other by replacing the solution in the reaction container 44 and by cleaning the reaction container 44. After replacing the solution in the reaction container 44 and cleaning the reaction container 44, when the substrate of the labeling enzyme is injected, the substrate is decomposed by the enzyme to form the thiol compound. After a certain time of the reaction, the formed thiol compounds are introduced into the reaction solution 50 in the measurement cell 49 using the thiol compound solution injector 48. The thiol compounds introduced into the reaction solution 50 in the measurement cell 49 are adsorbed on the gold electrode 51 formed on the insulated gate field-effect transistor 45 to form a self-assembled monolayer. As a result, the potential on the gold electrode 51 is changed. The measurement is performed by monitoring in real time the current between a source 54 and a drain 55 in the insulated gate field-effect transistor 45 which is changed before and after the injection of the thiol compounds produced by means of the thiol compound solution injector 48, and by recording the monitored value using the signal processing circuit 42 and the data processing unit 43. The rate of the adsorption of the thiol compounds on the gold electrode 51 is proportional to the concentration of the thiol compounds, that is, to the amount of the antibody-antigen-enzyme-labeled antibody complex. Accordingly, the amount of the bound labeling enzyme, that is, the amount of the antigen in the sample solution can be obtained by measuring the adsorption rate of the thiol compounds on the gold electrode 51.

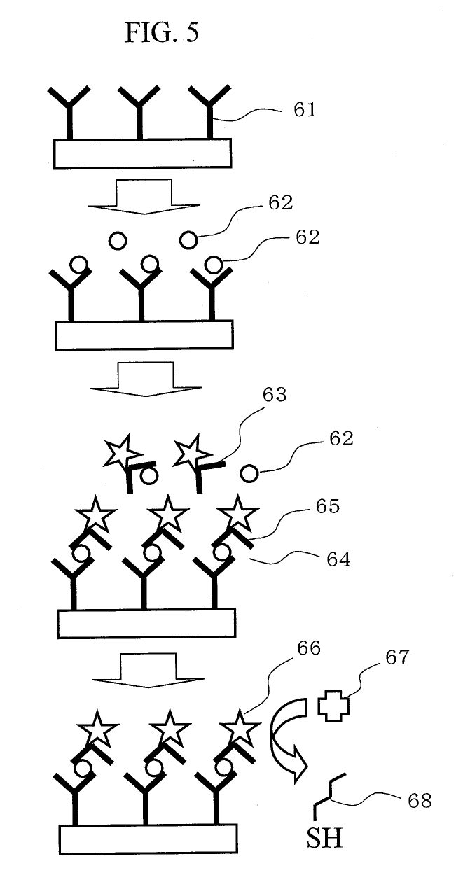

FIG. 5 shows a reaction flow in the immuno analyzer using the FET sensor according to the present invention.

In the immuno analysis, the amount of binding between antigen and antibody is measured using a specific binding reaction between the antigen and the antibody to obtain the amount of the antigen. In the present invention, using the sandwich method generally used in a conventional immuno analysis, the amount of the antigen is indirectly measured through, for example, enzyme which is used for labeling antibody. After reaction between antibody 61 previously immobilized to a solid phase and antigen 62 in a sample, enzyme-labeled antibody 63 is added thereto to form antibody-antigen-enzyme-labeled antibody complex 64. Then, bound enzyme-labeled antibody 65, the free enzyme-labeled antibody 63, and the free antigen 62 are separated from each other. A thiol compound 68 which is the reaction product of the cyclic reaction between enzyme 66 of the bound enzyme-labeled antibody and a substrate 67 can be measured by means of the FET sensor to obtain the amount of the antigen.

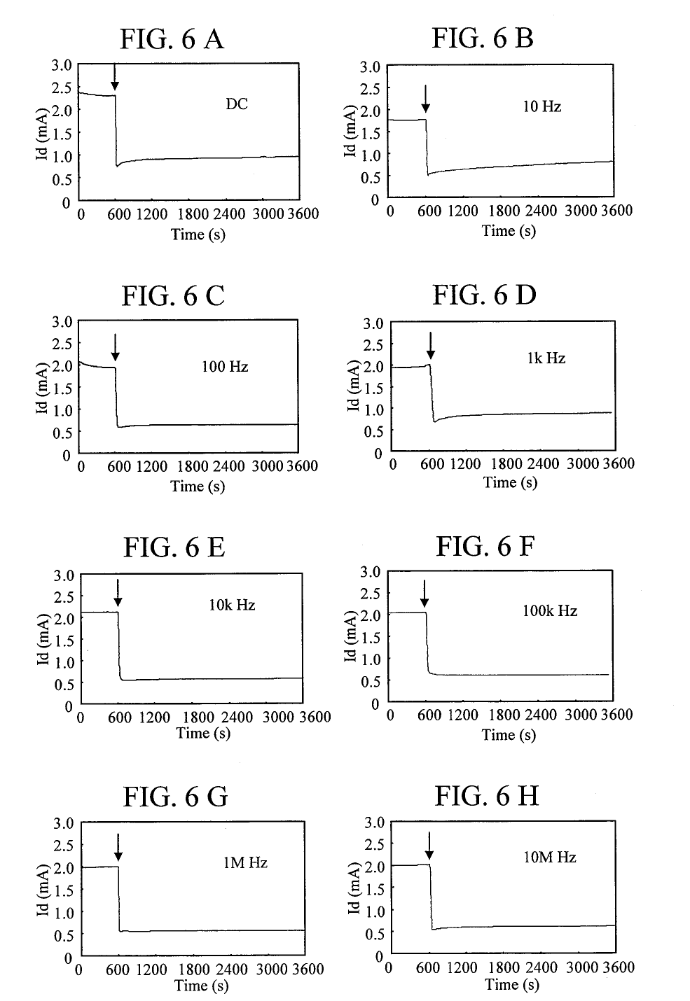

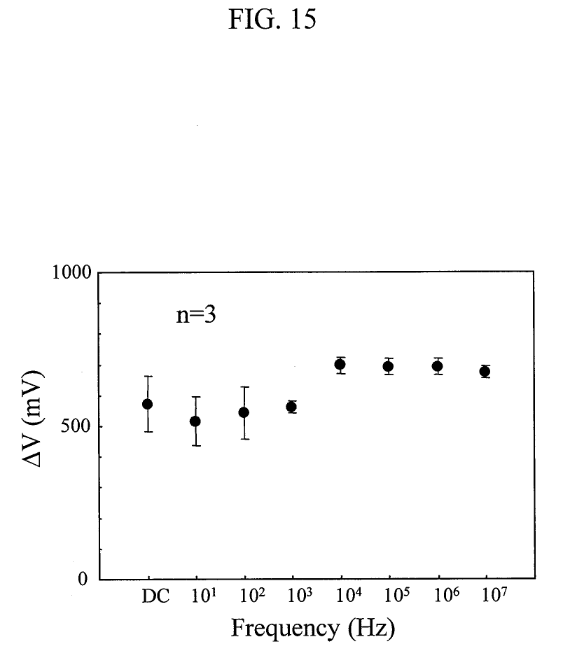

The effect of application of alternating voltage according to the present invention is described using other example. FIGS. 6A to 6H are drawings showing the changes with time of the drain current caused when the sample solution is introduced into the reaction solution 50 in the measurement cell 49 while applying the alternating voltage to the reference electrode 46 shown in FIG. 4. FIGS. 6B to 6H show the results obtained in a case of applying the alternating voltage at each frequency of 10 Hz, 100 Hz, 1 KHz, 10 KHz, 100 KHz, 1 MHz, and 10 MHz, respectively. FIG. 6A is a drawing showing the data obtained when applying direct current (DC) as a reference experiment to see the effect of application of alternating voltage. FIG. 15 is a drawing showing the relationship between the frequency of the applied the alternating voltage and the change in an interface potential (ΔV). The value was obtained by converting the change in the drain current to the change in the interface potential (ΔV). As the reference electrode 46, an Ag/AgCl reference electrode was used. Application of alternating voltage to the reference electrode 46 was performed using a center voltage of 100 mV and an amplitude voltage of 100 mV. As a reaction solution, 1.9 ml of 0.1 M Na₂SO₄ aqueous solution was used. For a sample solution, 1 mM 6-hydroxy-1-hexanethiol (6-HHT) aqueous solution was used as an alkane thiol compound. Measurement of the current-voltage properties of a transistor was performed using a semiconductor parameter analyzer (Agilent 4155C Semiconductor Parameter Analyzer).

The sample solution of 0.1 mL was introduced into the reaction solution after 600 seconds from starting measurement (shown by an arrow in each of the figures). After the introduction of the sample solution, the reduction in the drain current was seen in all cases. In the case of DC application, stability is so low that drift was large from the start of measurement before the introduction of the sample solution. In addition, after the introduction of the sample solution, the drain current value was once decreased, and increased again. It took 10 minutes or more to achieve stabilization. This tendency was seen in a case where the frequency was 1 KHz or less. Meanwhile, in a case where the frequency was 10 KHz or more, a drain current value was hardly increased after once decreased. It took a short time to achieve stabilization as compared to a case where the frequency was 1 KHz or less. The drain current had such a small drift as to be stable from the start of measurement before the introduction of the sample solution. The reason for the above results is considered to be because the introduction of the sample solution causes the surface potential on the gold electrode to be unstable, but the frequency of alternating current of 10 KHz or more applied to the reference electrode makes larger an effect of quickly restoring the irregularity of the surface potential on the gold electrode. Thus, the reaction process caused on the gold electrode can be measured with high accuracy by superimposing a high frequency of 10 KHz or more to the reference electrode during measurement.

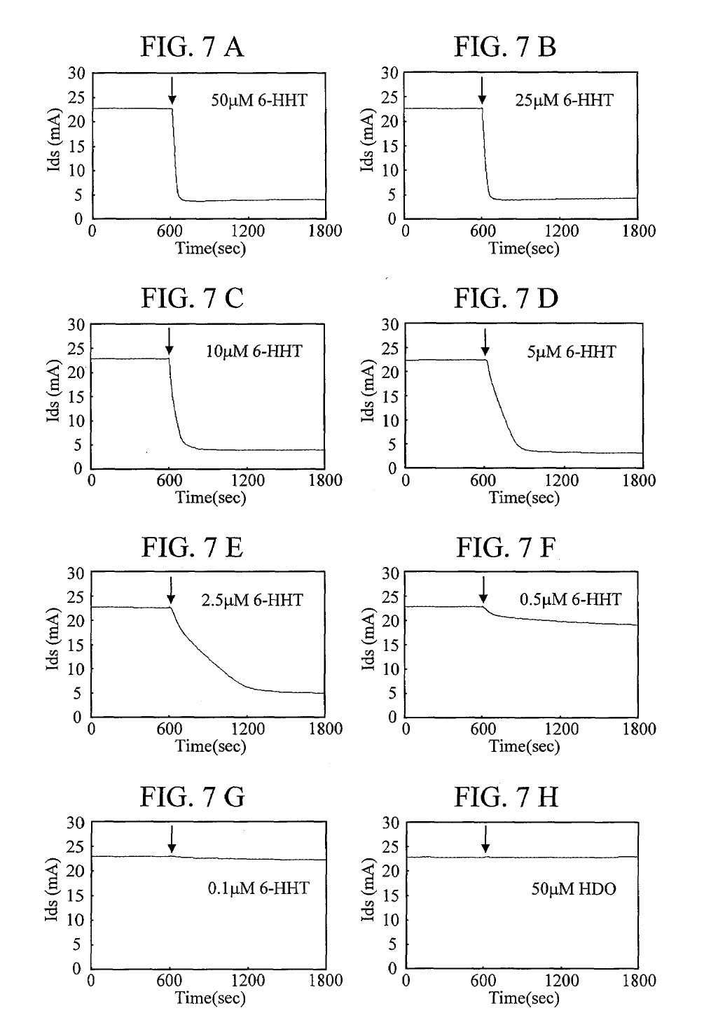

FIGS. 7A to 7H are drawings showing the results of the measurement of the sample solutions having different concentrations. For the sample solution, 6-HHT aqueous solution was used as an alkane thiol compound. FIGS. 7A to 7G show the results of the measurements with respect to the final concentration in the sample solution in the cases of 50 μM, 25 μM, 10 μM, 5 μM, 2.5 μM, 0.5 μM, and 0.1 μM, respectively. To see the effect of physical adsorption on the gold electrode, an HDO (hexanethidiol) aqueous solution was used as a reference experiment (final concentration: 50 μM). The arrow in the figure shows the time when the sample solution was introduced. As the reference electrode 46, an Ag/AgCl reference electrode was used. Application of alternating voltage on the reference electrode 46 was performed using a center voltage of 100 mV, an amplitude voltage of 100 mV, and a frequency 1 of MHz. 1.9 ml of 0.1 M Na₂SO₄ aqueous solution was used as a reaction solution. As shown in FIGS. 7A and 7B, in a case where the concentration of the reaction solution was 50 μM and 25 μM, drain current values became constant in a few seconds, and then adsorption reaction on the gold electrode was completed. As shown in FIGS. 7D and 7E, in a case where the concentration was 5 μM and 2.5 μM, it took about 5 minutes and about 10 minutes for reactions to be completed after the drain current became constant, respectively. Meanwhile, as shown in FIGS. 7F and 7G, in a case where the concentration was 0.5 μM and 0.1 μM, it took 1 hour or more for the drain current value to become constant. As shown in FIG. 7H, in the case of the HDO aqueous solution, the drain current value hardly changed. This shows that physical adsorption on the gold electrode has no effect.

As described above, the time until the drain current value becomes constant is varied depending on the concentration in the reaction solution. The concentration of the alkane thiol compounds in the sample solution can be found by measuring or estimating the time from the introduction of the sample solution until the drain current value becomes constant. Alternatively, instead of measuring or estimating the time from the introduction of the sample solution until the drain current value becomes constant, the concentration in the solution can also be found by using the amount of the change in the drain current value immediately after the introduction of the sample solution, and by using the adsorption rate of the alkane thiol compounds on the gold electrode. As the adsorption rate on the gold electrode, a function y=F(x) obtained by fitting the drain current value immediately after the introduction of the sample solution by, for example, a least-square method using a polynomial equation or an exponential function may be used. Alternatively, as an initial adsorption reaction rate immediately after the introduction of the sample solution, the differential value of F(x) or the changed amount in a certain time may be used.

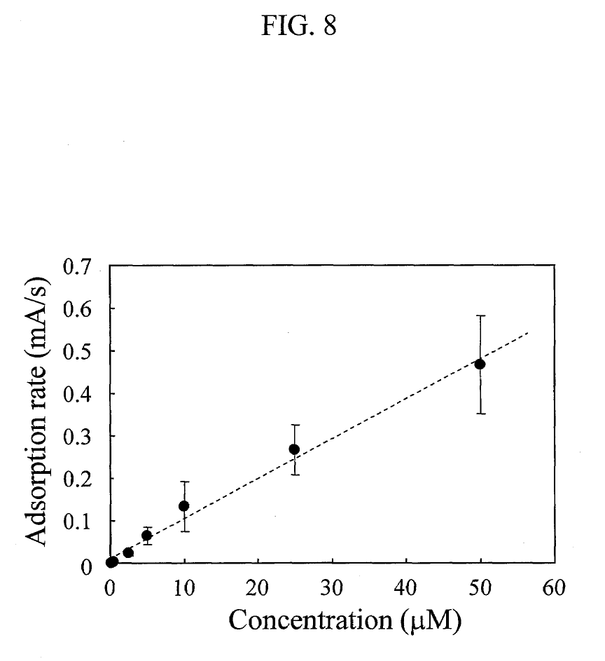

FIG. 8 is a drawing showing the relationship between the concentration in the sample solution and the adsorption rate on the gold electrodes which is the measurement result. Measurement was performed three times. The average thereof was plotted in the figure. The adsorption rate was determined by calculation performed using the tangent of a drain current curve immediately after the introduction of the sample solution as an initial rate. As shown in FIG. 8, the adsorption rate showed good linearity with the concentration in the sample solution. Thus, the concentration in the sample solution can quickly be obtained with accuracy from the adsorption rate on the gold electrode instead of using the measured time until the drain current value becomes constant and the time until the adsorption reaction is completed.

An enzyme immunoassay method using an apparatus of the present invention will be hereinafter described. In the present example, the amount of antigen was indirectly measured through, for example, the enzyme which was used for labeling antibody using a sandwich method generally used in a conventional immuno analysis. After the reaction between antibody previously immobilized on a plate and the antigen in the sample, enzyme-labeled antibody was added to form antibody-antigen-enzyme-labeled antibody complex. Then, the bound enzyme-labeled antibody, free enzyme-labeled antibody, and the free antigen were separated from one another. Subsequently, thiol compounds, which are the product of the cyclic reaction between the enzyme of the bound enzyme-labeled antibody and the substrate, was measured using an FET sensor. The sample and reagent used in the present example are listed below.

Immobilized antibody: Interleukin 1β antibody

Sample: Human plasma

Substance to be measured: Interleukin 1β

Enzyme-labeled antibody: acetylcholinesterase (AChE): Interleukin-1β Fab′Conjugate

Substrate: 2.5 mM acetylthiocholine

Reaction solution: 0.1 M phosphoate acid buffer (pH 7.4), 0.15 M NaCl, 1 mM EDTA

Note that, the reaction conditions and the concentration of the reagents used here are just an example, and can suitably be changed depending on the structure of an apparatus and a substance to be measured.

A measurement procedure is described below. First, sample solution of 100 μL (Human plasma), and enzyme-labeled antibody of 100 μL (AChE: Interleukin-1β Fab′ Conjugate) are put in the well of a plate on which antibody of Interleukin 1β is immobilized, and the plate is covered with a plastic film to react them at 4° C. overnight. Then, the solution in the well on the plate is discarded, and the well is cleaned with a wash buffer five or six times. An acetylthiocholin solution, which is the substrate of acetylcholinesterase, is put in each well to react it for about 30 minutes. By introducing a reaction solution containing the thiol compounds formed through the reaction into the reaction cell in which an FET sensor is immersed to measure the adsorption rate of the thiol compounds on the gold electrode, the concentration of the thiol compounds formed through the reaction is obtained. The concentration of the formed thiol compounds is proportional to the concentration of the enzyme of the antibody-antigen-enzyme-labeled antibody complex, and therefore the amount of antigen can be determined.

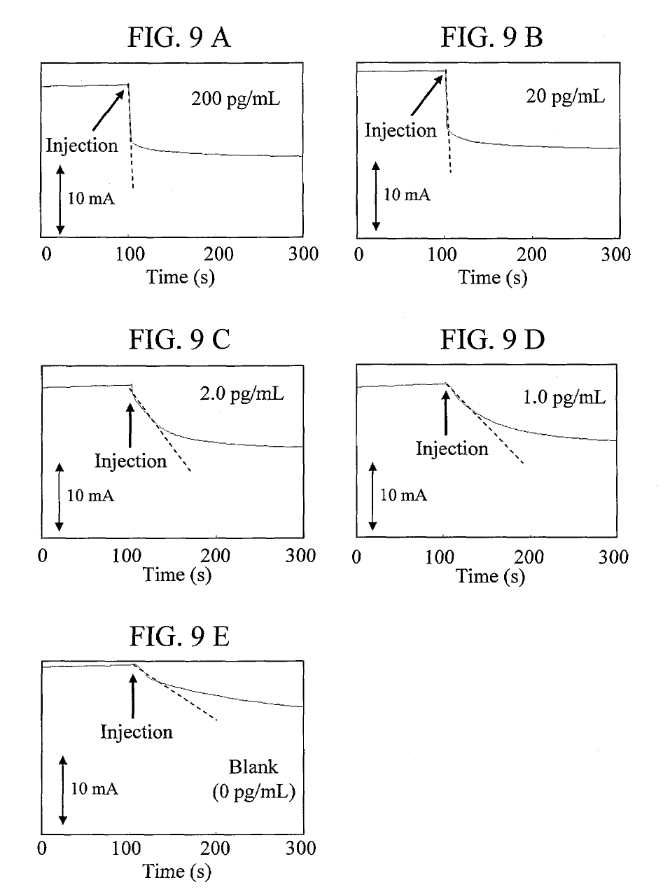

In measurement by means of an FET sensor, an Ag/AgCl reference electrode was used as a reference electrode. Alternating voltage having a center voltage of 100 mV, an amplitude voltage of 100 mV, and a frequency of 1 MHz was applied on the reference electrode. FIGS. 9A to 9E show the results of measurement by means of the FET sensor. The data shown in FIGS. 9A to 9D represents the measurement results of the sample having the concentration of 200 pg/mL, 20 pg/mL, 2.0 pg/mL, and 1.0 pg/mL of Interleukin 1β, which is antigen, respectively. The data shown in FIG. 9E represents the measurement result of a blank having the concentration of 0 pg/mL of the Interleukin 1β. In the present invention, the concentration of the Interleukin 1β in the sample was determined as the adsorption rate on the gold electrode. The adsorption rate was determined by calculation using the tangent (shown in dotted line in the drawing) of the drain current curve immediately after introducing the sample solution (shown with an arrow in the figure) as an initial rate.

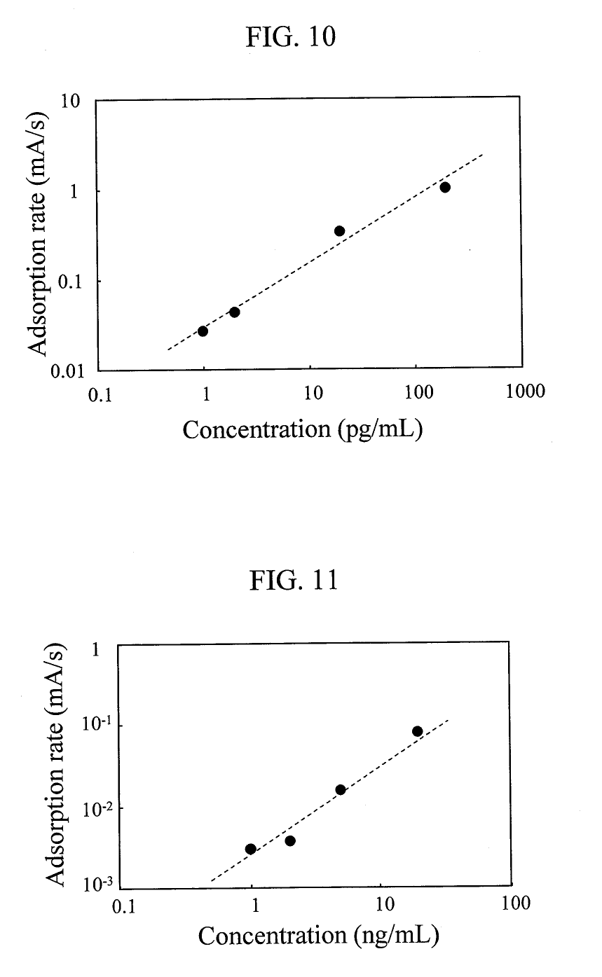

As a result, as shown in FIG. 10, the concentration in the sample solution and the adsorption rate showed such good linearity therebetween that measurement was possible up to 1.0 pg/mL. The value of this detection sensitivity is higher than that of the conventional method by one order or more. Thus, the concentration in the sample solution can be measured by means of an FET sensor with high sensitivity without using a spectrophotometer by measuring the rate of adsorption on the gold electrode of the thiol compounds formed through the enzyme reaction in the sandwich method generally used in the conventional immuno analysis method.

The following description is made about other example of an enzyme immunoassay method using the apparatus of the present invention. In the present example, thiol compounds, which are products of an enzyme immuno reaction, were measured in real time in the same reaction cell in the apparatus construction shown in FIG. 1. Note that, in measurement by means of an FET sensor, an Ag/AgCl reference electrode was used as a reference electrode. Alternating voltage having a center voltage of 100 mV, an amplitude voltage of 100 mV, and a frequency of 1 MHz was applied on the reference electrode. The sample and reagent used in the present example is listed below.

Immobilized antibody: Interleukin 1β antibody

Sample: Human plasma

Substance to be measured: Interleukin 1β

Enzyme-labeled antibody: acetylcholinesterase (AChE): Interleukin-1β Fab′ Conjugate

Substrate: 2.5 mM acetylthiocholine

Reaction solution: 0.1 M phosphate buffer (pH 7.4), 0.15 M NaCl, 1 mM EDTA

A measurement procedure is described below. First, an antibody immobilizing plate on which antibody of Interleukin 1β is immobilized is disposed in a reaction cell. The sample solution of 100 μL (Human plasma) and the enzyme-labeled antibody of 100 μL (AChE: Interleukin-1β Fab′ Conjugate) are added in the reaction cell, and the reaction cell is covered with a plastic film to react them at 4° C. overnight. Then, the solution in the reaction cell is discarded, and the cell is cleaned with a wash buffer five or six times. A reaction solution of 1.8 mL (0.1 M phosphate buffer (pH7.4), 0.15M NaCl, 1 mM EDTA) is added in the reaction cell to start measurement. Acetylthiocholine solution of 0.2 mL is introduced into the reaction cell after 600 seconds from starting measurement to form thiol compounds through enzyme reaction. The thiol compounds formed through the reaction are so adsorbed on the gold electrode that the drain current value of the FET sensor is changed. The production rate of the thiol compounds can be measured with accuracy by measuring the drain current value in real time. The production rate of the thiol compounds depends on the amount of the enzyme of antibody-antigen-enzyme-labeled antibody complex. Accordingly, the amount of bound labeling enzyme, that is, the amount of antigen can be obtained from the production rate of the thiol compounds.

In the present example, the concentration of the Interleukin 1β in the sample was figured out as the adsorption rate on the gold electrode. The adsorption rate was found by calculation using the tangent of the drain current curve immediately after introducing the sample solution as an initial rate. The results of measurement by means of the FET sensor are shown in FIG. 11. As shown in FIG. 11, the adsorption rate showed good linearity with the concentration in the sample solution.

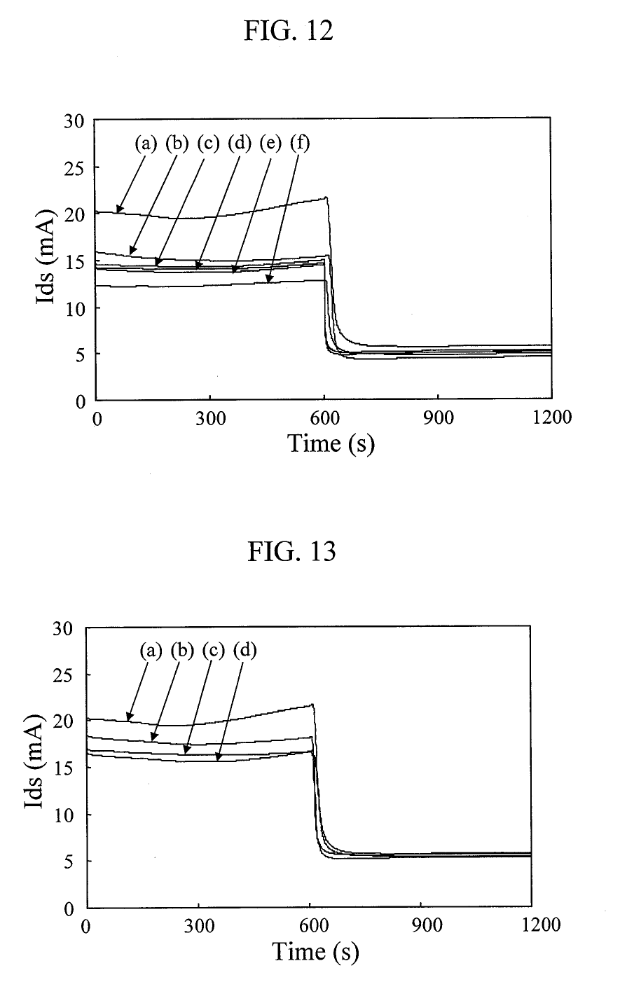

The following description is made about the effect of physical adsorption of straight-chain polymer on the gold electrode. Polyethylene glycols having different molecular weight were used as straight-chain polymer. FIG. 12 is a drawing showing the change with time in the drain current associated with the adsorption of the thiol compounds on the electrode on which the polyethylene glycol has been physically adsorbed. The curves (b) to (f) in FIG. 12 shows the data with respect to the gold electrodes coated with 0.5% aqueous solutions of polyethylene glycol having a molecular weight of 1000, 2000, 8000, 500000, and 2000000, respectively. The curve (a) in FIG. 12 shows a case where an untreated gold electrode was used as a reference. The thiol compounds used are 1 mM 6-HHT aqueous solution. 1.9 ml of 0.1 M Na₂SO₄ aqueous solution was used as a reaction solution. 0.1 mM 6-HHT aqueous solution was introduced after 600 seconds from starting measurement. An Ag/AgCl reference electrode was used as a reference electrode. Note that, to see the effect of the straight-chain polymer, application of alternating voltage to the reference electrode was not performed, but 100 mV of DC was applied. Measurement of current/voltage properties of the transistor was performed using a semiconductor parameter analyzer (Agilent 4155C Semiconductor Parameter Analyzer).

As a result, as shown in FIG. 12, the baseline significantly wavered up and down with respect to the untreated gold electrode before introducing the 6-HHT aqueous solution, but the baseline is stable in a case of the electrode on which polyethylene glycol is physically adsorbed. Particularly, in a case of the electrode on which polyethylene glycol having a molecular weight of 2000 or more is adsorbed, the wavering of the baseline disappears, and the baseline became stable.

FIG. 13 shows the measurement results in a case where dextran was used as another straight-chain polymer. FIG. 13 is a drawing showing the change with time in the drain current associated with the adsorption of the thiol compounds on the electrode on which dextran was physically adsorbed. The curves (b) to (d) in FIG. 13 show the data with respect to the gold electrodes which were coated with 0.5% solutions of dextran having a molecular weight of 40000, 90000, and 200000, respectively. The curve (a) in FIG. 13 shows the data with respect to the untreated gold electrode as a reference. The thiol compounds used are 1 mM 6-HHT aqueous solution. 1.9 ml of 0.1 M Na₂SO₄ aqueous solution was used as a reaction solution. 0.1 mM 6-HHT aqueous solution was introduced after 600 seconds from starting measurement. An Ag/AgCl reference electrode was used as a reference electrode. Note that, to see the effect of the straight-chain polymer, application of alternating voltage to the reference electrode was not performed, but DC of 100 mV was applied. Measurement of current/voltage properties of the transistor was performed using a semiconductor parameter analyzer (Agilent 4155C Semiconductor Parameter Analyzer). As a result, as in the case of FIG. 12, the baseline significantly wavered up and down with respect to the untreated gold electrode before introducing the 6-HHT aqueous solution, but the baseline became stable in a case of the gold electrode on which dextran was physically adsorbed.

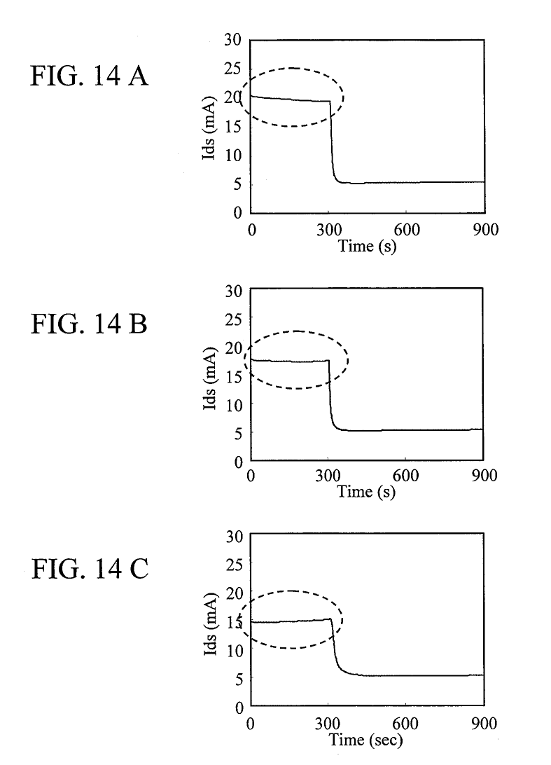

FIGS. 14A to 14C show the measurement results in a case of the combination of the gold electrode on which these straight-chain polymer are physically adsorbed and a high frequency superposition method. FIGS. 14A, 14B, and 14C correspond to an untreated gold electrode, a gold electrode which is coated with dextran (molecular weight: 2000000), and a gold electrode which is coated with polyethylene glycol (molecular weight: 500000), respectively. Note that, the 0.5% aqueous solution was used for the physical adsorption of the polymer on the gold electrode. The thiol compounds used are 1 mM 6-HHT aqueous solution. 1.9 ml of 0.1 M Na₂SO₄ aqueous solution was used as a reaction solution. 0.1 mM 6-HHT aqueous solution was introduced after 600 seconds from starting measurement. An Ag/AgCl reference electrode was used as a reference electrode. Alternating voltage having a center voltage of 100 mV, an amplitude voltage of 100 mV, and a frequency of 1 MHz was applied to the reference electrode. Measurement of current/voltage properties of the transistor was performed using a semiconductor parameter analyzer (Agilent 4155C Semiconductor Parameter Analyzer).

As a result, as shown in FIGS. 14A to 14C by a circle in a dotted line, the baseline was stable with a small wavering before introducing 6-HHT aqueous solution in a case of the untreated gold electrode. In a case of the electrode which was coated with dextran or polyethylene glycol, the wavering of the baseline was hardly seen. From this, a synergy effect was seen between the coating of straight-chain polymer and a high frequency superposition method.

What is claimed is:

1. A measurement apparatus comprising:
a container which contains antibody to a substance to be measured;
sample solution supply means which supplies, to the container, a sample solution containing the substance to be measured;
enzyme-labeled antibody supply means supplying enzyme-labeled antibody in which enzyme used for producing thiol compounds in the container and the antibody to the substance to be measured are bound to each other;
substrate supply means which supplying the substrate for the enzyme;
a field-effect transistor;
an electrode connected to a gate of the field-effect transistor with a wire and being in contact with the solution in the container;
a reference electrode being in contact with the solution in the container;
a power supply for applying a voltage between the electrode and the reference electrode; and
a detection section for detecting the output of the field-effect transistor.

2. The measurement apparatus as set forth in claim 1, wherein the power supply applies an alternating voltage of 10 kHz or more.

3. The measurement apparatus as set forth in claim 1, wherein the antibody is immobilized on a solid phase.

4. The measurement apparatus as set forth in claim 1, wherein the antibody is immobilized on the electrode.

5. The measurement apparatus as set forth in claim 1, wherein the electrode is made of a noble metal.

6. The measurement apparatus as set forth in claim 1, wherein straight-chain polymer is physically adsorbed on the electrode.

7. The measurement apparatus as set forth in claim 1, comprising a processing section for calculating the amount of change in the output of the field-effect transistor, after the substrate supply means supplies the substrate.

8. The measurement apparatus as set forth in claim 1, comprising a processing section for calculating the initial rate of change in the output of the field-effect transistor, after the substrate supply means supplies the substrate.

9. A measurement apparatus comprising:
a container into which a measurement solution containing thiol compounds is introduced;
a field-effect transistor;
an electrode connected to a gate of the field-effect transistor with a wire and being in contact with the measurement solution in the container;
a reference electrode being in contact with the measurement solution in the container;
a power supply for applying a voltage between the electrode and the reference electrode; and
a detection section for detecting the output of the field-effect transistor.

10. The measurement apparatus as set forth in claim 9, wherein the power supply applies alternating voltage of 10 kHz or more.

11. The measurement apparatus as set forth in claim 9, wherein the electrode is made of a noble metal.

12. The measurement apparatus as set forth in claim 9, wherein straight-chain polymer is physically adsorbed on the electrode.

13. The measurement apparatus as set forth in claim 9, comprising a processing section for calculating the amount of change in the output of the field-effect transistor, after the measurement solution containing the thiol compounds is supplied.

14. The measurement apparatus as set forth in claim 9, comprising a processing section for calculating the initial rate of change in the output of the field-effect transistor, after the measurement solution containing the thiol compounds is supplied.

15. An element for analysis comprising:
a field-effect transistor; and
an electrode made of a noble metal, on the surface of which straight-chain polymer is physically adsorbed,
wherein a gate of the field-effect transistor and the electrode are connected to each other with an electroconductive wire.

16. The element for analysis as set forth in claim 15, wherein the noble metal is any of gold and silver.

17. The element for analysis as set forth in claim 15, wherein the straight-chain polymer is any of dextran and polyethylene glycol.
